(19) United States
(12) Patent Application Publication
(10) Pub. No.: US 2023/0343438 A1
(43) Pub. Date: Oct. 26, 2023

(54) SYSTEMS AND METHODS FOR AUTOMATIC IMAGE ANNOTATION

(71) Applicant: SHANGHAI UNITED IMAGING INTELLIGENCE CO., LTD., SHANGHAI (CN)

(72) Inventors: Meng Zheng, Cambridge, MA (US); Qin Liu, Chapel Hill, NC (US); Srikrishna Karanam, Bangalore (IN); Ziyan Wu, Lexington, MA (US)

(73) Assignee: SHANGHAI UNITED IMAGING INTELLIGENCE CO., LTD., Shanghai (CN)

(21) Appl. No.: 17/726,369

(22) Filed: Apr. 21, 2022

(51) Int. Cl. G16H 30/40; G06N 3/04; G06F 40/169

(52) U.S. Cl. CPC G16H 30/40; G06N 3/04; G06F 40/169

(57) ABSTRACT

Described herein are systems, methods, and instrumentalities associated with automatic image annotation. The annotation may be performed based on one or more manually annotated first images of an object and a machine-learned (ML) model trained to extract first features from the one or more first images. To automatically annotate a second, un-annotated image of the object, the ML model may be used to extract second features from the second image, determine information that may be indicative of the characteristics of the object in the second image based on the first and second features, and generate an annotation of the object for the second image using the determined information. The images may be obtained from various sources including, for example, sensors and/or medical scanners, and the object of interest may include anatomical structures such as organs, tumors, etc. The annotated images may be used for multiple purposes including machine learning.

### BACKGROUND

Having annotated data is crucial to the training of machine-learning (ML) models or artificial neural networks. Current data annotation relies heavily on manual work, and even when computer-based tools are provided, they still require a tremendous amount of human effort (e.g., mouse clicking, drag-and-drop, etc.). This strains resources and often leads to inadequate and/or inaccurate results. Accordingly, it is highly desirable to develop systems and methods to automate the data annotation process such that more data may be obtained for ML training and/or verification.

### SUMMARY

Described herein are systems, methods, and instrumentalities associated with automatic image annotation. An apparatus capable of performing the image annotation task may include one or more processors that are configured to obtain a first image of an object and a first annotation of the object, and determine, using a machine-learned (ML) model (e.g., implemented via an artificial neural network) and the first annotation, a first plurality of features (e.g., a first feature vector) from the first image. The first annotation may be generated with human intervention (e.g., at least partially) and may identify the object in the first image, for example, through an annotation mask. The one or more processors of the apparatus may be further configured to obtain a second, un-annotated image of the object and determine, using the ML model, a second plurality of features (e.g., a second feature vector) from the second image. Using the first plurality of features extracted from the first image and the second plurality of features extracted from the second image, the one or more processors of the apparatus may be configured to generate, automatically (e.g., without human intervention), a second annotation of the object that may identify the object in the second image.

In examples, the one or more processors of the apparatus described above may be further configured to provide a user interface for generating the first annotation. In examples, the one or more processors of the apparatus may be configured to determine the first plurality of features from the first image by applying respective weights to the pixels of the first image in accordance with the first annotation. The weighted imagery data thus obtained may then be processed based on the ML model to extract the first plurality of features. In examples, the one or more processors of the apparatus may be configured to determine the first plurality of features from the first image by extracting preliminary features from the first image using the ML model and then applying respective weights to the preliminary features in accordance with the first annotation to obtain the first plurality of features.

In examples, the one or more processors of the apparatus described herein may be configured to generate the second annotation by determining one or more informative features based on the first plurality of features extracted from the first image and the second plurality of features extracted from the second image, and generating the second annotation based on the one or more informative features. For instance, the one or more processors may be configured to generate the second annotation of the object by aggregating the one or more informative features (e.g., a set of features common to both the first and the second plurality of features) into a numeric value and generating the second annotation based on the numeric value. In examples, this may be accomplished by backpropagating a gradient of the numeric value through the ML model and generating the second annotation based on respective gradient values associated with one or more pixel locations of the second image.

The first and second images described herein may be obtained from various sources including, for example, from a sensor that is configured to capture the images. Such a sensor may include a red-green-blue (RGB) sensor, a depth sensor, a thermal sensor, etc. In other examples, the first and second images may be obtained using a medical imaging modality such as a computer tomography (CT) scanner, a magnetic resonance imaging (MRI) scanner, an X-ray scanner, etc. and the object of interest may be anatomical structure such as a human organ, a human tissue, a tumor, etc. While embodiments of the present disclosure may be described using medical images as examples, those skilled in the art will appreciate that the disclosed techniques may also be used to process other types of data.

### BRIEF DESCRIPTION OF THE DRAWINGS

A more detailed understanding of the examples disclosed herein may be had from the following description, given by way of example in conjunction with the accompanying drawing.

FIG. 1 is a diagram illustrating an example of automatic image annotation in accordance with one or more embodiments of the disclosure provided herein.

FIG. 2 is a diagram illustrating example techniques for automatically annotating a second image based on an annotated first image in accordance with one or more embodiments of the disclosure provided herein.

FIG. 3 is a flow diagram illustrating example operations that may be associated with automatic annotation of an image in accordance with one or more embodiments of the disclosure provided herein.

FIG. 4 is a flow diagram illustrating example operations that may be associated with training a neural network to perform one or more of the tasks described herein.

FIG. 5 is a block diagram illustrating example components of an apparatus that may be configured to perform the image annotation tasks described herein.

### DETAILED DESCRIPTION

The present disclosure is illustrated by way of example, and not by way of limitation, in the figures of the accompanying drawings.

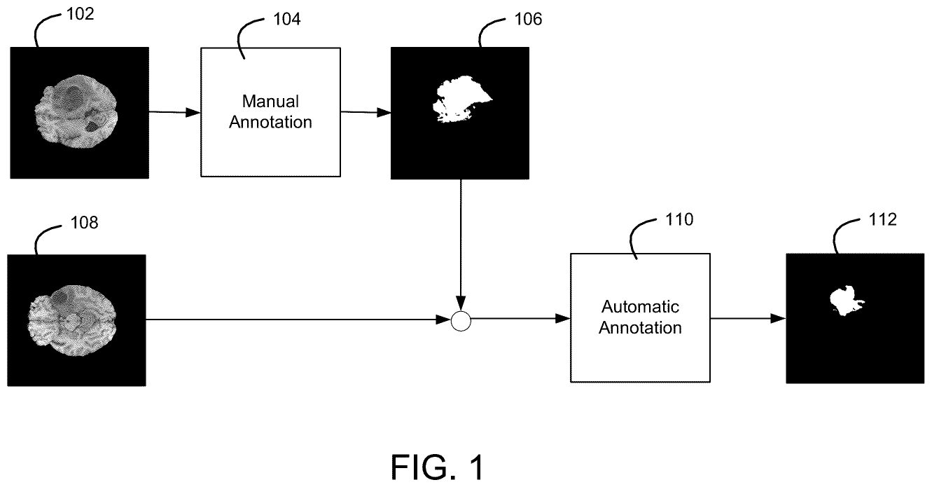

FIG. 1 illustrates an example of automatic data annotation in accordance with one or more embodiments of the present disclosure. The example will be described in the context of medical images but those skilled in the art will appreciate that the disclosed techniques may also be used to process other types of images or data including, for example, alphanumeric data. As shown in FIG. 1, image 102 (e.g., a first image) may include a medical image captured using an imaging modality (e.g., X-ray, computer tomography (CT), or magnetic resonance imaging (MRI)) and the image may include an object of interest such as a human organ, a human tissue, a tumor, etc. In other examples, image 102 may include an image of an object (e.g., including a person) that may be captured by a sensor. Such a sensor may be installed in or around a facility (e.g., a medical facility) and may include, for example, a red-green-blue (RGB) sensor, a depth sensor, a thermal sensor, etc.

Image 102 may be annotated for various purposes. For example, the image may be annotated such that the object of interest in the image may be delineated (e.g., labeled or marked up) from the rest of the image and used as ground truth for training a machine learning (ML) model (e.g., an artificial neural network) for image segmentation. The annotation may be performed through annotation operations 104, which may involve human effort or intervention. For instance, annotation operations 104 may be performed via a computer-generated user interface (UI), and by displaying image 102 on the UI and requiring a user to outline the object in the image using an input device such as a computer mouse, a keyboard, a stylus, a touch screen, etc. The user interface and/or input device may, for example, allow the user to create a bounding box around the object of interest in image 102 through one or more of the following actions: clicks, taps, drags-and-drops, clicks-drags-and-releases, scratches, drawing motions, etc. These annotation operations may result in a first annotation 106 of the object of interest being created (e.g., generated). The annotation may be created in various forms including, for example, an annotation mask that may include respective values (e.g., Booleans or decimals having values between 0 and 1) for the pixels of image 102 that may indicate whether (e.g., based on a likelihood or probability) each of the pixels belongs to the object of interest or an area outside of the object of interest (e.g., a background area).

The annotation (e.g., first annotation 106) created through operations 104 may be used to annotate (e.g., automatically) one or more other images of the object of interest. Image 108 of FIG. 1 shows an example of such an image (e.g., a second image), which may include the same object of interest as image 102 but with different characteristics (e.g., different contrasts, different resolutions, different viewing angles, etc.). As will be described in greater detail below, image 108 may be annotated automatically (e.g., without human intervention) through operations 110 based on first annotation 106 and/or respective features extracted from image 102 and image 108 to generate second annotation 112 that may mark (e.g., distinguish) the object of interest in image 108. Similar to first annotation 106, second annotation 108 may be generated in various forms including, for example, an annotation mask described herein. And once generated, annotation 108 may be presented to a user (e.g., via the UI described herein) so that further adjustments may be made to refine the annotation. In examples, the adjustments may be performed using the UI described herein and by executing one or more of the following actions: clicks, taps, drags-and-drops, clicks-drags-and-releases, scratches, drawing motions, etc. In examples, adjustable control points may be provided along an annotation contour created by annotation 112 (e.g., on the UI described herein) to allow the user to adjust the annotation contour by manipulating the adjustable control points (e.g., by dragging and dropping one or more of the control points to various new locations on the display screen).

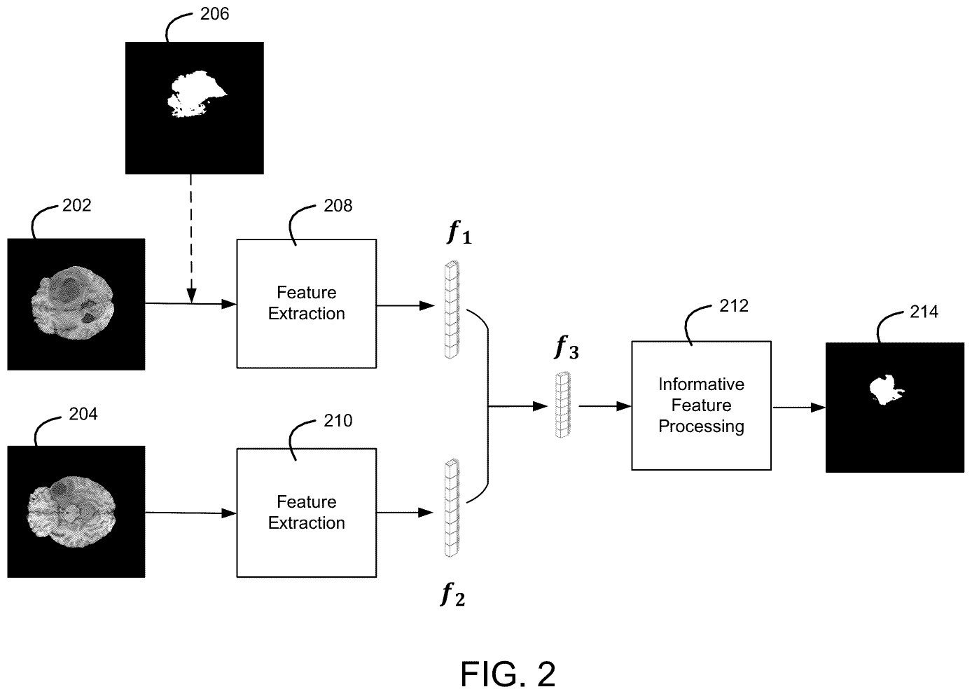

FIG. 2 illustrates example techniques for automatically annotating a second image 204 of an object based on an annotated first image 202 of the object. The first image may be annotated with human intervention, for example, using the UI and the manual annotation techniques described herein. Based on the first image and the manually obtained annotation (e.g., first annotation 206 shown in FIG. 2, which may be in the form of an annotation mask as described herein), a first plurality of features, f₁, may be determined from the first image at 208 using a machine-learned (ML) feature extraction model that may be trained (e.g., offline) for identifying characteristics of an image that may be indicative of the location of an object of interest in the image. The ML feature extraction model may be learned and/or implemented using an artificial neural network such as a convolutional neural network (CNN). In examples, such a CNN may include an input layer configured to receive an input image and one or more convolutional layers, pooling layers, and/or fully-connected layers configured to process the input image. The convolutional layers may be followed by batch normalization and/or linear or non-linear activation (e.g., such as a rectified linear unit or ReLU activation function). Each of the convolutional layers may include a plurality of convolution kernels or filters with respective weights, the values of which may be learned through a training process such that features associated with an object of interest in the image may be identified using the convolution kernels or filters upon completion of the training. These extracted features may be down-sampled through one or more pooling layers to obtain a representation of the features, for example, in the form of a feature vector or a feature map. In some examples, the CNN may also include one or more un-pooling layers and one or more transposed convolutional layers. Through the un-pooling layers, the network may up-sample the features extracted from the input image and process the up-sampled features through the one or more transposed convolutional layers (e.g., via a plurality of deconvolution operations) to derive an up-scaled or dense feature map or feature vector. The dense feature map or vector may then be used to predict areas (e.g., pixels) in the input image that may belong to object of interest. The prediction may be represented by a mask, which may include a respective probability value (e.g., ranging from 0 to 1) for each image pixel that indicates whether the image pixel may belong to object of interest (e.g., having a probability value above a preconfigured threshold) or a background area (e.g., having a probability value below a preconfigured threshold).

First annotation 206 may be used to enhance the completeness and/or accuracy of the first plurality of features f₁ (e.g., which may be obtained as a feature vector or feature map). For example, using a normalized version of annotation 206 (e.g., by converting probability values in the annotation mask to a value range between 0 and 1), first image 202 (e.g., pixel values of the first image 202) may be weighted (e.g., before the weighted imagery data is passed to the ML feature extraction neural network 208) such that pixels belonging to the object of interest may be given larger weights during the feature extraction process. As another example, the normalized annotation mask may be used to apply (e.g., inside the feature extraction neural network) respective weights to the features (e.g., preliminary features) extracted by the feature extraction neural network at 208 such that features associated with the object of interest may be given larger weights in the first plurality of features f₁ produced by the feature extraction neural network.

Referring back to FIG. 2, second image 204 (e.g., an un-annotated image comprising the same object as first image 202) may also be processed using an ML feature extraction model (e.g., the same ML feature extraction neural network used to process first image 202) to determine a second plurality of features f₂ at 210. The second plurality of features f₂ may be represented in the same format as the first plurality of features f₁ (e.g., a feature vector) and/or may have the same size as f₁ The two sets of features may be used jointly to determine a set of informative features f₃ that may be indicative of the pixel characteristics of the object of interest in first image 202 and/or second image 204. For instance, informative features f₃ may be obtained by comparing features f₁ and f₂, and selecting the common features between f₁ and f₂. One example way of accomplishing this task may be to normalize feature vectors f₁ and f₂ (e.g., such that both vectors have values ranging from 0 to 1), compare the two normalized vectors (e.g., based on (f₁-f₂)), and selecting corresponding elements in the two vectors that have a value difference smaller than a predefined threshold as the informative features f₃.

In examples, the second plurality of features f₂ extracted from second image 204 and/or the informative features f₃ may be further processed at 212 to gather information (e.g., from certain dimensions of f₂) that may be used to automatically annotate the object of interest in second image 204. For example, based on informative features f₃, an indicator vector having the same size as feature vectors f₁ and/or f₂ may be derived in which elements that correspond to informative features f₃ may be given a value of 1 and the remaining elements may be given a value of 0. A score may then be calculated to aggregate of the informative features f₃ and/or the informative elements of feature vector f₂. Such a score may be calculated, for example, by conducting an element-wise multiplication of the indicator vector and feature vector f₂. Using this calculated score, annotation 214 (e.g., a second annotation) of the object of interest may be automatically generated for second image 204, for example, by backpropagating a gradient of the score through the feature extraction neural network (e.g., the network used at 210) and determining pixel locations (e.g., spatial dimensions) that may correspond to the object of interest based on respective gradient values associated with the pixel locations. For instance, pixel locations having positive gradient values during the backpropagation (e.g., these pixel locations may make positive contributions to the desired results) may be determined to be associated with the object of interest and pixel locations having negative gradient values during the backpropagation (e.g., these pixel locations may not make contributions or may make negative contributions to the desired results) may be determined to be not associated with the object of interest. Annotation 214 of the object of interest may then be generated for the second image based on these determinations, for example, as a mask determined based on a weighted linear combination of the feature maps obtained using the feature extraction network (e.g., the gradients may operate as the weights in the linear combination).

The annotation (e.g., annotation 214) generated using the techniques described herein may be presented to a user, for example, through an user interface (e.g., the UI described above) so that further adjustments may be made by the user to refine the annotation. For example, the user interface may allow the user to adjust the contour of annotation 214 by executing one or more of the following actions: clicks, taps, drags-and-drops, clicks-drags-and-releases, scratches, drawing motions, etc. Adjustable control points may be provided along the annotation contour and the user may be able to change the shape of the annotation by manipulating one or more of these control points (e.g., by dragging and dropping the control points to various new locations on the display screen).

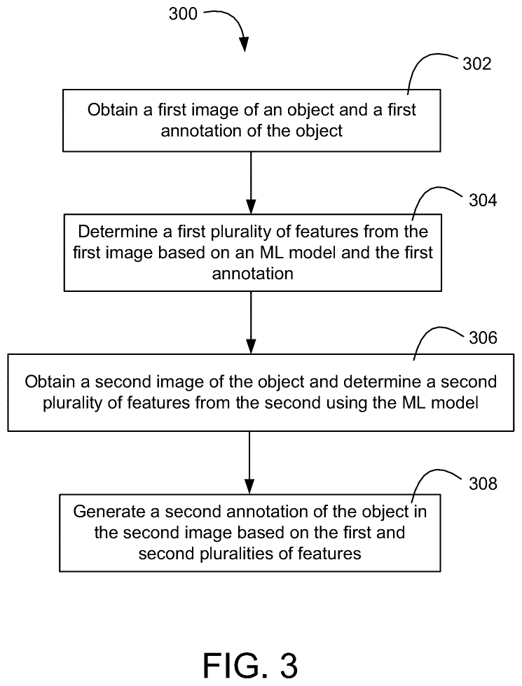

FIG. 3 illustrates example operations 300 that may be associated with the automatic annotation of a second image of an object of interest based on an annotated first image of the object of interest. As shown, the first image and a first annotation (e.g., an annotation mask) of the first image may be obtained at 302. The first image may be obtained from different sources including, for example, a sensor (e.g., an RGB, depth, or thermal sensor), a medical imaging modality (e.g., CT, MRI, X-ray, etc.), a scanner, etc., and the first annotation may be generated with human intervention (e.g., manually, semi-manually, etc.). Based on the first image and/or the first annotation, a first plurality of features may be extracted from the first image using a machined-learned feature extraction model (e.g., trained and/or implemented using a feature extraction neural network). These features may be indicative of the characteristics (e.g., pixel characteristics such as edges, contrast, etc.) of the object of interest in the first image and may be used to identify the object in other images. For instance, at 306, a second image of the object of interest may be obtained, which may be from the same source as the first image, and a second plurality of features may be extracted from the second image using the ML model. The second plurality of features may then be used, in conjunction with the first plurality of features, to automatically generate a second annotation that may mark (e.g., label) the object of interest in the second image. The second annotation may be generated at 308, for example, by identifying informative features (e.g., common or substantially similar features) based on the first and second images (e.g., based on the first plurality of features and the second plurality of features), aggregating information associated with the informative features (e.g., by calculating a score or numeric value based on the common features), and generating the second annotation based on the aggregated information (e.g., by backpropagating a gradient of the calculated score or numeric value through the feature extraction neural network).

The first and/or second annotation described herein may be refined by a user, and a user interface (e.g., a computer generated user interface) may be provided for accomplishing the refinement. In addition, it should be noted that the automatic annotation techniques disclosed herein may be based on and/or further improved by more than one previously generated annotated image (e.g., which may be manually or automatically generated). For example, when multiple annotated images are available, an automatic annotation system or apparatus as described herein may continuously update the information that may be extracted from these annotations and use the information to improve the accuracy of the automatic annotation.

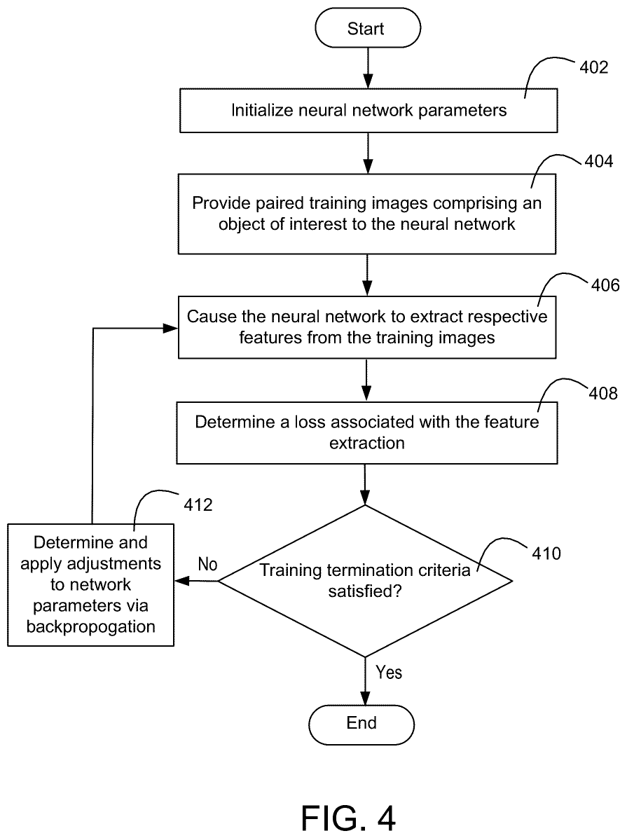

FIG. 4 illustrates example operations that may be associated with training a neural network (e.g., the feature extraction neural network described herein with respect to FIG. 2) to perform one or more of tasks described herein. As shown, the training operations may include initializing the parameters of the neural network (e.g., weights associated with the various filters or kernels of the neural network) at 402. The parameters may be initialized, for example, based on samples collected from one or more probability distributions or parameter values of another neural network having a similar architecture. The training operations may further include providing a pair of training images at least one of which may comprise an object of interest to the neural network at 404, and causing the neural network to extract respective features from the pair of training images at 406.

At 408, the extracted features may be compared to determine a loss, e.g., using one or more suitable loss functions (e.g., mean squared errors, L1/L2 losses, adversarial losses, etc.). The determined loss may be evaluated at 410 to determine whether one or more training termination criteria have been satisfied. For instance, a training termination criterion may be deemed satisfied if the loss(es) described above is below (or above) a predetermined thresholds, if a change in the loss(es) between two training iterations (e.g., between consecutive training iterations) falls below a predetermined threshold, etc. If the determination at 410 is that the training termination criterion has been satisfied, the training may end. Otherwise, the loss may be backpropagated (e.g., based on a gradient descent associated with the loss) through the neural network at 412 before the training returns to 406.

The pair of training images provided to the neural network may belong to the same category (e.g., both images may be brain MRI images containing a tumor) or the pair of images may belong to different categories (e.g., one image may be a normal MRI brain image and the other image may be an MRI brain image containing a tumor). As such, the loss function used to train the neural network may be selected such that feature differences between a pair of images belonging to the same category may be minimized and feature differences between a pair of images belonging to different categories may be maximized.

For simplicity of explanation, the training steps are depicted and described herein with a specific order. It should be appreciated, however, that the training operations may occur in various orders, concurrently, and/or with other operations not presented or described herein. Furthermore, it should be noted that not all operations that may be included in the training process are depicted and described herein, and not all illustrated operations are required to be performed.

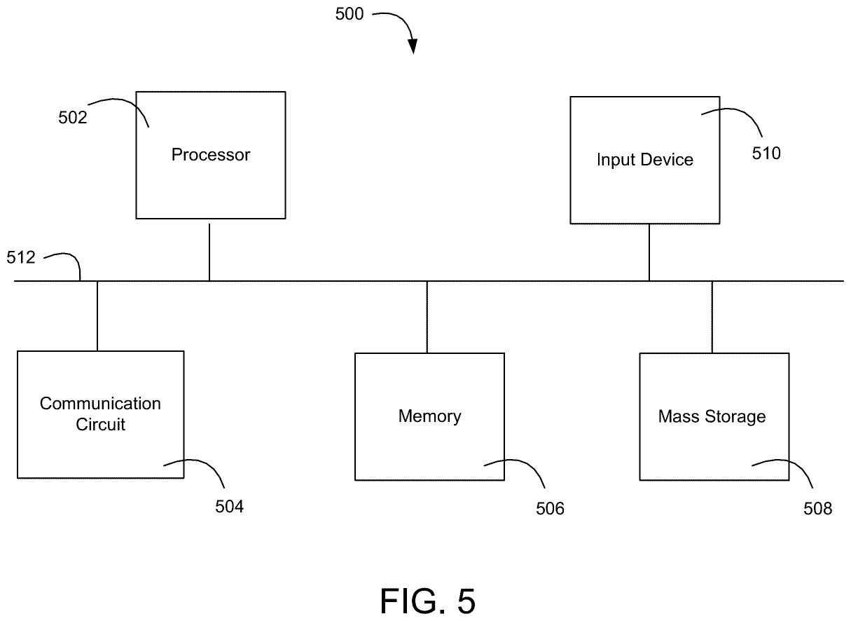

The systems, methods, and/or instrumentalities described herein may be implemented using one or more processors, one or more storage devices, and/or other suitable accessory devices such as display devices, communication devices, input/output devices, etc. FIG. 5 is a block diagram illustrating an example apparatus 500 that may be configured to perform the automatic image annotation tasks described herein. As shown, apparatus 500 may include a processor (e.g., one or more processors) 502, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 500 may further include a communication circuit 504, a memory 506, a mass storage device 508, an input device 510, and/or a communication link 512 (e.g., a communication bus) over which the one or more components shown in the figure may exchange information.

Communication circuit 504 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 506 may include a storage medium (e.g., a non-transitory storage medium) configured to store machine-readable instructions that, when executed, cause processor 502 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 508 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 502. Input device 510 may include a keyboard, a mouse, a voice-controlled input device, a touch sensitive input device (e.g., a touch screen), and/or the like for receiving user inputs to apparatus 500.

It should be noted that apparatus 500 may operate as a standalone device or may be connected (e.g., networked, or clustered) with other computation devices to perform the functions described herein. And even though only one instance of each component is shown in FIG. 5, a skilled person in the art will understand that apparatus 500 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. An apparatus, comprising:
one or more processors configured to:
obtain a first image of an object and a first annotation of the object, wherein the first annotation identifies the object in the first image;
determine, using a machine-learned (ML) model and the first annotation of the object, a first plurality of features from the first image;
obtain a second image of the object;
determine, using the ML model, a second plurality of features from the second image; and
generate a second annotation of the object based on the first plurality of features and the second plurality of features, wherein the second annotation identifies the object in the second image.

2. The apparatus of claim 1, wherein the first annotation is generated with human intervention and the second annotation is generated automatically based on the first annotation.

3. The apparatus of claim 2, wherein the one or more processors are further configured to provide a user interface for generating the first annotation.

4. The apparatus of claim 1, wherein the one or more processors being configured to determine the first plurality of features from the first image using the ML model and the first annotation of the object comprises the one or more processors being configured to apply respective weights to pixels of the first image based on the first annotation to obtain weighted imagery data and extract the first plurality of features based on the weighted imagery data using the ML model.

5. The apparatus of claim 1, wherein the one or more processors being configured to determine the first plurality of features from the first image using the ML model and the first annotation of the object comprises the one or more processors being configured to obtain preliminary features from the first image using the ML model, apply respective weights to the preliminary features based on the first annotation to obtain weighted preliminary features, and determine the first plurality of features based on the weighted preliminary features.

6. The apparatus of claim 1, wherein the one or more processors being configured to generate the second annotation based on the first plurality of features and the second plurality of features comprises the one or more processors being configured to identify one or more informative features based on the first plurality of features and the second plurality of features, and generate the second annotation based on the one or more informative features.

7. The apparatus of claim 6, wherein the one or more processors are configured to aggregate the one or more informative features into a numeric value and generate the second annotation based on the numeric value.

8. The apparatus of claim 7, wherein the one or more processors are configured to backpropagate a gradient of the numeric value through the ML model and generate the second annotation based on respective gradient values associated with one or more pixel locations of the second image.

9. The apparatus of claim 1, wherein at least one of the first image or the second image is obtained from a sensor configured to capture images of the object.

10. The apparatus of claim 9, wherein the sensor includes a red-green-blue (RGB) sensor, a depth sensor, or a thermal sensor.

11. The apparatus of claim 1, wherein the ML model is implemented using an artificial neural network.

12. A method for automatically annotating an image, the method comprising:
obtaining a first image of an object and a first annotation of the object, wherein the first annotation identifies the object in the first image;
determining, using a machine-learned (ML) model and the first annotation of the object, a first plurality of features from the first image;
obtaining a second image of the object;
determining, using the ML model, a second plurality of features from the second image; and
generating a second annotation of the object based on the first plurality of features and the second plurality of features, wherein the second annotation identifies the object in the second image.

13. The method of claim 12, wherein the first annotation is generated with human intervention and wherein the second annotation is generated automatically based on the first annotation.

14. The method of claim 13, wherein further comprising providing a user interface for generating the first annotation.

15. The method of claim 12, wherein determining the first plurality of features from the first image using the ML model and the first annotation of the object comprises applying respective weights to pixels of the first image based on the first annotation to obtain weighted imagery data and extracting the first plurality of features based on the weighted imagery data using the ML model.

16. The method of claim 12, wherein determining the first plurality of features from the first image using the ML model and the first annotation of the object comprises obtaining preliminary features from the first image using the ML model, applying respective weights to the preliminary features based on the first annotation to obtain weighted preliminary features, and determining the first plurality of features based on the weighted preliminary features.

17. The method of claim 12, wherein generating the second annotation based on the first plurality of features and the second plurality of features comprises identifying one or more informative features based on the first plurality of features and the second plurality of features, and generating the second annotation based on the one or more informative features.

18. The method of claim 17, wherein generating the second annotation of the object based on the one or more informative features comprises aggregating the one or more informative features into a numeric value and generating the second annotation based on the numeric value.

19. The method of claim 18, wherein generating the second annotation based on the numeric value comprises backpropagating a gradient of the numeric value through the ML model and generating the second annotation based on respective gradient values associated with one or more pixel locations of the second image.

20. The method of claim 12, wherein the ML model is implemented using an artificial neural network.
